(19) United States
(12) Patent Application Publication
(10) Pub. No.: US 2020/0181254 A1
(43) Pub. Date: Jun. 11, 2020

(54) VACCINE COMPOSITION COMPRISING A MUTANT OF HUMAN INTERLEUKIN-15

(71) Applicant: CENTRO DE INGENIERÍA GENÉTICA Y BIOTECNOLOGÍA, La Habana (CU)

(72) Inventors: Yunier RODRIGUEZ ALVAREZ, Artemisa (CU); Yanelys MORERA DÍAZ, La Habana (CU); Haydee GERONIMO PEREZ, La Habana (CU)

(21) Appl. No.: 16/474,913

(22) PCT Filed: Dec. 20, 2017
(86) PCT No.: PCT/CU2017/050008

§ 371 Date: Jun. 28, 2019

(30) Foreign Application Priority Data
Dec. 30, 2016 (CU) 2016-0194

(51) Int. Cl. C07K 16/24; A61K 39/00; A61P 29/00

(52) U.S. Cl. CPC C07K 16/244; A61K 39/00114; A61K 2039/55538; A61K 2039/505; A61P 29/00

(57) ABSTRACT

Vaccine composition containing a mutant of the human Interleukin-15 (IL-15). The use of said mutant polypeptide of human IL-15 to manufacture a medicament for the active immunotherapy of diseases associated with IL-15 overexpression, including some autoimmune diseases and hematological malignancies. Administration to an individual that needs it of a therapeutically effective amount of the vaccine, that comprises the mutant polypeptide of the human IL-15 of the invention, constitutes a method for the therapy of IL-15 over-expressing related diseases.

### CROSS-REFERENCE TO RELATED APPLICATIONS

This application is a U.S. National Phase of, and Applicant claims priority from, International Patent Application No. PCT/CU2017/050008, filed Dec. 20, 2017, which claims priority from CU 2016/0194, filed Dec. 30, 2016, each of which is incorporated herein by reference in its entirety.

### FIELD OF TECHNIQUE

The present invention relates to the branch of biotechnology, immunology and the pharmaceutical industry, in particular with active immunization using polypeptides comprising a mutated Interleukin-15 (IL-15), for the treatment of diseases associated with IL-15 overexpression.

### STATE OF THE PRIOR ART

The cytokine known as IL-15 is a 14-15 KDa glycoprotein, which was simultaneously described by two groups as a T cells-activating growth factor (Grabstein K H., et al. Science 1994; 264: 965-8; Burton, J D., et al. Proc. Natl. Acad. Sci. USA 1994; 91: 4935-9). IL-15 mRNA is widely expressed in cells and tissues, however, it is difficult to find the protein in these cells or in the cells supernatant due to a strong post-transcriptional control of its expression at the translational level and the intracellular trafficking (Bamford R N., et al. J. Immunol. 1998; 160: 4418-26; Kurys G., et al. J Biol Chem. 2000; 275: 30653-9).

The IL-15 biological effects are mediated through its binding to a cell membrane receptor composed of three subunits: α, β, and γ. The IL-15Rα is a specific, high-affinity (Kd 10⁻¹¹) IL-15 binding subunit, β subunit is shared with IL-2 and γ subunit is a common receptor for several cytokines, such as: IL-2; IL-4; IL-7; IL-9; IL-15; IL-21 (Bamford R N., et al. Proc. Natl. Acad. Sci. USA 1994; 91: 4935-9; Giri J G., et al. EMBO J. 1995; 14: 3654-63).

The IL-15 is an immunostimulatory cytokine that promotes proliferation and functional activity of T, B and NK cells (Giri J G., et al. EMBO J. 1994; 13: 2822-30), activates neutrophils and modifies monokine secretion (Girard D., et al. Blood 1996; 88: 3176-84; Alleva D G., et al. J. Immunol. 1997; 159: 2941-51). Also, this cytokine induces CD56 NK cells proliferation and acts, together with IL-12, inducing IFN-γ and tumor necrosis factor (TNF)-α (Ross M E. and Caligiuri M A. Blood 1997; 89: 910-8; Fehniger T A., et al. Transplant Proc. 1999; 31: 1476-8).

Unregulated expression or overexpression of IL-15 has been associated with the development of several diseases, including Crohn's disease (Kirman I. and Nielsen O H. Am. J. Gastroenterol. 1996; 91(9): 1789-94), psoriasis (Rückert R., et al., J. Immunol. 2000; 165: 2240-50), leukemia (Yamada Y., et al. Blood 1998; 91: 4265-72; Yamada Y. and Kamihira S. Leuk Lymphoma 1999; 35(1-2):37-45) and Rheumatoid arthritis (AR) (McInnes I B. Immunology Today 1998; 19: 75-9).

Previous studies have suggested that IL-15 precede TNF-α in the cytokine cascade, acting as an important factor in the migration of T cells to synovial fluid (Feldmann M., et al. Annu Rev Immunolo 1996; 14: 397-440; McInnes I B., et al. Nat Med. 1997; 3: 189-95). Additionally, Ziolkowska et al. reported that IL-15 induces IL-17 expression at joints from RA patients and it stimulates release by synoviocytes of several inflammatory mediators such as IL-6, IL-8, GM-CSF, and prostaglandin E₂; suggesting an important role for IL-15 in the RA pathogenesis (Ziolkowska M., et al. J Immunol. 2000; 164: 2832-8).

The use of IL-15 antagonist molecules has been shown to be effective in animal models. Ruchatz et al. generated a soluble fragment from the alpha subunit of the murine receptor (IL-15Rα) and demonstrated that this fragment inhibited collagen-induced arthritis in DBA/1 mice (Ruchatz H., et al. J. Immunol. 1998; 160: 5654-60).

Other IL-15 antagonist molecules have been patented, including mutants of IL-15 in one or more amino acids residues and monoclonal antibodies against mature IL-15 that prevents signal transduction through its receptor (U.S. Pat. Nos. 6,001,973, 6,177,079, 6,168,783, 6,013,480). Although their use is described in the aforementioned patent documents, there are few studies in the literature that support its effectiveness.

On May 2002, Genmab announced the clinical study phase I and II of an anti-IL-15 antibody (HuMax-IL-15) (U.S. Pat. No. 6,177,079). Although the treatment was well tolerated, no significant efficacy was demonstrated in a phase II study involving 110 patients with RA (Baslund B., et al. Arthritis & Rheumatism. 2005; 52: 2686-92). Other studies have been performed with a chimeric protein consisting of an IL-15 mutant and the Fc fragment of an immunoglobulin, which binds to cells expressing the IL-15 receptor. This fusion protein, called CRB-15, has been shown to be effective in a murine model of collagen induced arthritis, where CRB-15 decreases the incidence and severity of the disease, and prevents the progression of arthritis once established (Ferrari-Lacraz S., et al. J Immunol. 2004; 173(9):5818-26).

In 2003, Santos et al. proposed a vaccination method that included active immunization with IL-15 obtained from Escherichia coli (International Patent Application Publication Number WO 2004/032956). However, the protein obtained in that host is biologically active as it promotes the proliferation of the CTLL-2, an IL-2/IL-15 dependent cell line (Santos-Savio A., et al. Biotecnol. Apl. 2000; 17: 221-4). This element is a disadvantage, taking into account that small amounts of the immunogen that are released and enter into circulation would trigger an unwanted response. This cytokine, under physiological conditions, is known to be a memory T cell growth factor (Kanai T., et al. J Immunol. 1996; 157(8): 3681-7; Kanegane H. and Tosato G. Blood 1996; 88(1): 230-5). The inflammatory process in RA is characterized by activation and recruitment of T cells to the synovial (Wilkinson P C. and Liew F Y. Exp Med. 1995; 181: 1255-9), so immunization with an active IL-15 molecule may contribute to the development of the disease.

It remains of interest to obtain vaccines for active immunotherapy in diseases associated with IL-15 overexpression, which offer therapeutic advantages on current therapies aimed at the treatment of these types of diseases.

### BRIEF DESCRIPTION OF DRAWINGS

FIG. 1A. Purity of IL-15Mut. RP-HPLC chromatographic profile of IL-15Mut obtained in E. coli, used as antigen of the vaccine preparation.

FIG. 1B. Purity of IL-15Mut. RP-HPLC chromatographic profile of recombinant IL-15 obtained in E. coli, used as antigen of the vaccine preparation.

FIG. 2A. Evaluation of IL-15Mut biological activity by CTLL-2 cell proliferation assay. Cell proliferation induced by serial dilutions of native IL-15 (R&D), IL-15Mut and recombinant IL-15 obtained in E. coli.

FIG. 2B. Evaluation of IL-15Mut biological activity by CTLL-2 cell proliferation assay. Cell proliferation curve in the presence of serial dilutions of IL-15Mut with a fixed concentration of native IL-15.

FIG. 3A. Evaluation by ELISA of the anti-IL-15 antibody response in monkeys immunized with IL-15Mut or IL-15, using Aluminum hydroxide as adjuvant. The graphs represent the mean values of the antibody titers of the three animals in each experimental group, corresponding to 15 days after the second and the third immunization.

FIG. 3B. Evaluation by ELISA of the anti-IL-15 antibody response in monkeys immunized with IL-15Mut or IL-15, using Montanide™ ISA-51 as adjuvant. The graphs represent the mean values of the antibody titers of the three animals in each experimental group, corresponding to 15 days after the second and the third immunization.

FIG. 4A. Evaluation in CTLL-2 cells of the neutralizing capacity of the individual sera after the third immunization using Alum as adjuvant. IL-15 group. The line called native IL-15 dilution represents the highest value of cell proliferation and the line named minimum indicates the minimum proliferation value for CTLL-2 cells grown in cytokine-free culture medium.

FIG. 4B. Evaluation in CTLL-2 cells of the neutralizing capacity of the individual sera after the third immunization using Alum as adjuvant. IL-15Mut group at dose of 200 μg. The line called native IL-15 dilution represents the highest value of cell proliferation and the line named minimum indicates the minimum proliferation value for CTLL-2 cells grown in cytokine-free culture medium.

FIG. 4C. Evaluation in CTLL-2 cells of the neutralizing capacity of the individual sera after the third immunization using Alum as adjuvant. IL-15Mut group at dose of 350 μg. The line called native IL-15 dilution represents the highest value of cell proliferation and the line named minimum indicates the minimum proliferation value for CTLL-2 cells grown in cytokine-free culture medium.

FIG. 4D. Evaluation in CTLL-2 cells of the neutralizing capacity of the individual sera after the third immunization using Alum as adjuvant. Placebo group. The line called native IL-15 dilution represents the highest value of cell proliferation and the line named minimum indicates the minimum proliferation value for CTLL-2 cells grown in cytokine-free culture medium.

FIG. 5. Evaluation in CTLL-2 cells of the neutralizing capacity of the pool of sera from animals immunized with IL-15Mut and recombinant IL-15. The neutralizing effects of each group, 15 days after the second and third immunizations, using aluminum hydroxide as adjuvant is shown. The line called native IL-15 dilution represents the highest value of cell proliferation and the line named minimum indicates the minimum proliferation value for CTLL-2 cells grown in cytokine-free culture medium.

FIG. 5. Evaluation in CTLL-2 cells of the neutralizing capacity of the pool of sera from animals immunized with IL-15Mut and recombinant IL-15. The neutralizing effects of each group, 15 days after the second and third immunizations, using Montanide™ as adjuvant is shown. The line called native IL-15 dilution represents the highest value of cell proliferation and the line named minimum indicates the minimum proliferation value for CTLL-2 cells grown in cytokine-free culture medium.

### INCORPORATION OF SEQUENCE LISTING

Incorporated herein by reference in its entirety is the Sequence Listing for the application. The Sequence Listing is disclosed on a computer-readable ASCII text file titled, “Vaccine Composition Comprising a Mutant of Human Interleukin-15”, created on Sep. 18, 2019. The Sequence Listing .txt file is 3.12 KB (3,200 bytes) in size.

### DETAILED DESCRIPTION OF THE INVENTION

The present invention solves the above-mentioned problem by providing a vaccine composition characterized in that it contains a polypeptide which comprises a mutant of the human IL-15 identified as SEQ ID No. 1, at least one vaccine adjuvant and pharmaceutically acceptable excipients or carriers.

The IL-15 gene was isolated from lipopolysaccharide (LPS)-activated monocytes and amplified by polymerase chain reaction (PCR), using oligonucleotides specific for mutation in the sequence of IL-15. Specifically, the Asp 8 and Gin 108 were replaced by Ser. The mutated polypeptide was obtained with the modifications in the amino acid residues mentioned above, hereinafter referred to as IL-15Mut, having a purity of greater than 95%.

In one embodiment of the invention, the vaccine composition comprises a polypeptide containing the identified IL-15 mutant (IL-15Mut) SEQ ID No. 1, which is obtained by recombinant deoxyribonucleic acid (DNA) technology. To obtain this molecule, hosts which are known to those skilled in the art, such as bacteria, yeasts, mammalian cells, etc. may be employed.

In one embodiment of the invention, the IL-15Mut polypeptide was obtained from E. coli, so it is not glycosylated, unlike the autologous protein present in the human. This mutated cytokine is not biologically active in the cell proliferation assay in CTLL-2, since it is not able to induce the growth of these cells, which are dependent on IL-15. In CTLL-2 cells, IL-15Mut competes with native IL-15 for receptor binding, but does not induce signaling by having mutated the amino acids Asp 8 and Gin 108, which are key residues for the interaction with the β and γ subunits of the receptor. This element is an advantage over the method proposed by Santos et al. (International Patent Application Publication No. WO 2004/032956), regarding a lower occurrence of adverse events, with respect to the pro-inflammatory activity of this cytokine, in the treated patients.

In one aspect of the present invention, the polypeptide comprising the IL-15 mutant identified as SEQ ID No. 1, which is part of a vaccine composition, is a fusion polypeptide. In a particular embodiment, the fusion polypeptide comprising the IL-15 mutant identified as SEQ ID No. 1 also comprises T epitopes, at a protein concentration of 200 μg.

In the present invention, it was designed an IL-15 mutant which corresponds to SEQ ID No. 1 obtaining by recombinant DNA technology; with structural characteristics different from the natural cytokine with respect to the localization of the disulfide bridges. This element promotes the development of the immune response by exposure of cryptic or immunodominant epitopes.

To demonstrate the concept proposed in this invention non-human primates (Chlorocebus sabaeus) were used; taking into account that this is the species with the highest homology, in the amino acid sequence of IL-15, with respects to human (˜97% of identity) (Grabstein K H., et al. Science 1994; 264(5161):965-8). It was demonstrated that active immunization of such primates, with a vaccine composition comprising the IL-15Mut polypeptide identified as SEQ ID No. 1, using aluminum hydroxide (alternatively called Alumina) and the oily adjuvant Montanide™ ISA-51, elicits a response of antibodies that inhibit the biological activity of native IL-15 in CTLL-2 cells. This cell line, used for the neutralization assays, expresses the three subunits of the IL-15 receptor, and is dependent on this cytokine to proliferate (Eisenman J., et al. Cytokine 2002; 20(3): 121-9). In this assay, the sera showed inhibition the proliferation in a dose-dependent manner. This response is higher when compared to the group of animals immunized with the polypeptide identified as SEQ ID No. 2 (IL-15 protein obtained in E. coli). The results also shown that immunization with a higher dose (350 μg) of the polypeptide of SEQ ID No.1, generates a higher neutralizing antibody response from the 15 days after the second immunization. This aspect is an advantage, being that the desired effect is achieved with a lower number of administrations of the vaccine composition of the present invention.

Considering the high percentage of similarity between the mutated polypeptide of SEQ ID No. 1 and simian IL-15, it is unexpected that immunization with this mutated protein generates a neutralizing antibody response against native IL-15. However, the present invention demonstrates that immunization with the mutated polypeptide identified as SEQ ID No. 1 generates neutralizing autoantibodies against autologous IL-15. In addition, it is surprising that the neutralizing antibody response generated by administration of that polypeptide (SEQ ID No. 1) is greater than the obtained by active immunization with recombinant IL-15 (SEQ ID No. 2).

The vaccine composition of the present invention may be administered by several routes, as it is known to those skilled in the art. The amount of vaccine antigen in the composition may vary, depending on the disease to be treated. In one embodiment of the invention, the amount of antigen (polypeptide comprising a mutant of the human IL-15 identified as SEQ ID No. 1) is in the range of 200-500 μg per vaccine dose. In the course of patient immunization, repeated doses may be administered and the compositions may contain vaccine adjuvants which are well known in the current vaccination, such as aluminum salts and water-in-oil emulsions, and others which are in development; in order to generate in the patient the levels of antibodies against IL-15 that are required.

The invention also discloses the use of a polypeptide comprising a mutant of human IL-15, identified as SEQ ID No. 1, to manufacture a medicament for the active immunotherapy of a disease associated with IL-15 overexpression. It is well known to those skilled in this field of the art those diseases involving aberrant expression or over-expression of IL-15.

In one embodiment of the invention, the polypeptide comprising a human IL-15 mutant identified as SEQ ID No. 1, is used to manufacture a medicament for the active immunotherapy of an autoimmune disease selected from the group consisting of the RA, Crohn's disease, ulcerative colitis and psoriasis.

In another aspect of the invention, said polypeptide is used for the active immunotherapy of a disease associated with IL-15 overexpression, such as hematological malignancies. In a particular embodiment, the hematological neoplasia is leukemia or a lymphoma.

The invention also provides a method for the treatment of diseases associated with IL-15 overexpression. It is characterized by comprising the administration to an individual that needs it of a therapeutically effective amount of a vaccine composition that comprises a polypeptide comprising a mutant of the human IL-15 identified as SEQ ID No. 1. As stated above, said vaccine composition is suitable for administration to humans, and generates a neutralizing antibody response against autologous IL-15.

The method of the invention, comprising active immunization with the IL-15Mut (of SEQ ID No. 1) in a formulation suitable to generate neutralizing autoantibodies against IL-15, enables the neutralization of unregulated amounts of this cytokine in patients with autoimmune diseases and hematological malignancies. This method is superior to that proposed in the International Patent Application No. WO 2004/032956, taking into account that the antigen used for the active immunization lacks biological activity.

The method of the invention is useful for the treatment of diseases associated with IL-15 overexpression, including RA, psoriasis and inflammatory bowel diseases. Particularly in these pathologies, the generation of antibodies that neutralize the IL-15 activity is advantageous. In patients affected by these diseases, the active immunotherapy method of the invention may be combined with the administration of other medicaments, such as anti-inflammatories or antagonists of other cytokines. These medicaments include, for example, steroid drugs, such as corticosteroids and traditional disease-modifying antirheumatic drugs (e.g. methotrexate).

The method of the invention is also useful for the treatment of diseases such as hematological malignancies, in which leukemia and lymphomas are found. In this context, the active immunotherapy employing the vaccine composition of the invention may be combined with the administration, to the individual requiring it, of at least one pharmaceutically acceptable cytostatic.

The method of the invention is advantageous, since the levels of antibodies generated in the patient itself are more stable over time than those administered by passive immunization. On the other hand, the frequency of application of the vaccine compositions comprising the IL-15 mutant is much less than in a passive immunization with antibodies against the cytokine (in the method of this invention they are spaced for a longer time interval). In addition, the amounts of protein per dose, and the amount of administrations required for vaccination, are lower than those required for treatment with other IL-15 antagonist molecules, which implies a lower cost of production and higher adherence to treatment.

Immunization with the polypeptide identified as SEQ ID No. 1 generates antibodies with a greater neutralizing capacity than those obtained in the group immunized with the recombinant protein of SEQ ID No. 2. The use of an antigen that is not biologically active results in a lower frequency of undesirable events in patients treated with the vaccine containing it.

### DETAILED DISCLOSURE OF EMBODIMENTS/EXAMPLES OF REALIZATION

Example 1. Obtaining polypeptide IL-15Mut in E. coli DNA coding for human IL-15 was isolated from LPS-activated monocytes and it was amplified by PCR (annealing temperature 60° C. for 25 cycles), using specific primers for the mutation of Asp⁸ and Gln¹⁰⁸ by Ser in the IL-15 sequence (primer 5′ CAT GCC ATG GCA AAC TGG GTG AATGTA ATA AGT TCT TTG AAA (SEQ ID No. 3) and primer 3′ C GGGATCCCG TTA AGA AGT GTT GAT GAA CAT AGA GAC AAT (SEQ ID No. 4). The PCR product was digested with Nco I/BamH I (Promega, USA) and cloned into an E. coli expression vector. The IL-15Mut, obtained as inclusion bodies, was solubilized with urea 8M. The renaturation process was carried out on a 1.6×40 cm column (GE Healthcare Life Sciences, USA) packed with Sephadex G-25 Fine (Pharmacia Biotech, Sweden) and equilibrated with 0.1 M Tris buffer and 0.15 M NaCl; pH 8.0 at the rate flow of 7 mL/min. The collected sample was applied to a 1.6×10 cm column (GE Healthcare, USA) packed with Q Sepharose Fast Flow (GE Healthcare, USA), using a linear gradient of elution from 0.2 M to 0.5 M NaCl, at a flow rate of 2 mL/min. The sample eluted with 0.5 M NaCl was applied to a C₄ column (1×25 cm, 10 μm, Vydac, USA), at a flow of 1.0 mL/min. Proteins were separated using a mobile phase containing 0.1% trifluoroacetic acid (TFA) and acetonitrile HPLC grade); employing the following gradient: 0-30% in 5 min; 30% for 10 min; 30-40% in 5 min; 40-50% in 30 min; 50-60% in 60 min; 60-80% in 5 min at a flow of 2.5 mL/min. Proteins separation were monitored at 226 nm. The Bradford method was employed to determine the total protein concentration, according to manufacturer's instructions. Non-mutated IL-15 protein was also obtained recombinantly in E. coli, as previously described (Santos-Savio A., et al. Biotecnol. Apl. 2000; 17: 221-4).

### Example 2. Determination of IL-15Mut Purity by RP-HPLC

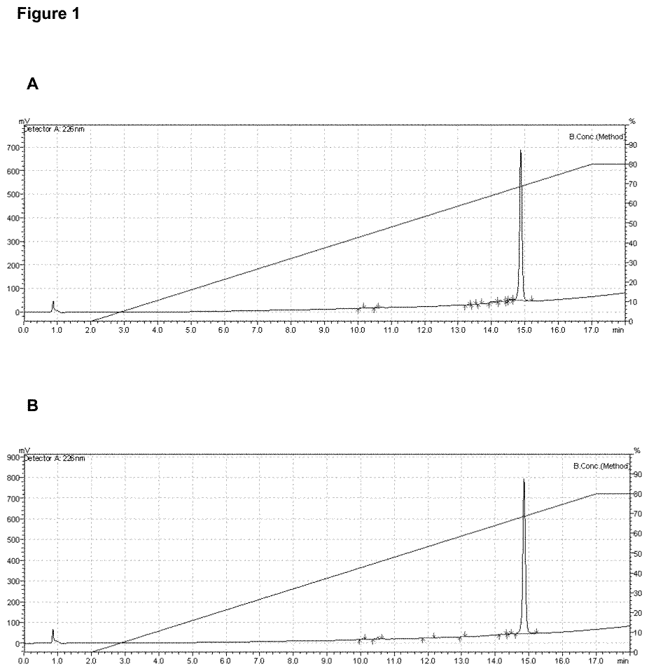

Samples collected from the major RP-HPLC purification peak were checked to estimate percent purity. The protein concentration was determined by the modified Lowry method, with the set of reagents “Modified Lowry Protein Assay Kit” (Thermo Scientific, USA), according to manufacturer's instructions. The RP-HPLC analysis was conducted with 50 μg of purified proteins on Chromolith Performance C₈ column (4.6×100 mm, 2 μm, Merck, USA) using a gradient from 0 to 80% of AcN/0.1% TFA in 15 min, at a flow rate of 2.5 mL/min. The detection wavelength was set at 226 nm. The purity was determined using the program Image J v1.32. IL-15Mut was obtained with a purity of 96%, as seen in FIG. 1; while non-mutated recombinant IL-15 was obtained with 98% of purity.

### Example 3. Evaluation of the IL-15Mut Biological Activity in CTLL-2 Cell Line

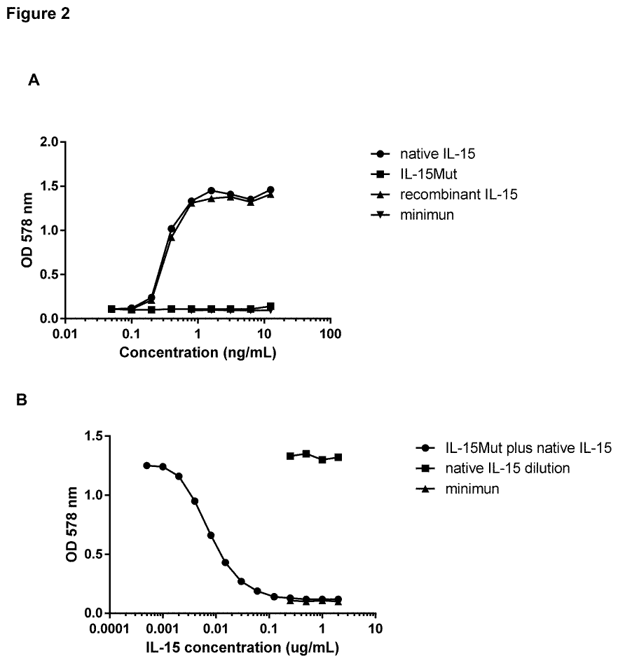

In order to evaluate the biological activity of IL-15Mut, the proliferation assay in CTLL-2 cell line was performed. Biological activity was measured by stimulation of CTLL-2 cells proliferation, using mitochondrial staining with 3-(4,5-dimethylthiazol-2-yl)-2,5-diphenyltetrazolium bromide (MTT, 3-[4, 5-Dimethylthiazol-2-yl]-2,5-diphenyltetrazolium bromide) (Mossman T J. Immunol. Methods 1983; 65(1-2): 55-63), following the procedure described below. Twofold serial dilutions of recombinant IL-15, native IL-15 (R&D, USA) and IL-15Mut (starting concentration 25 ng/mL) were performed in 96-well plates (Costar, USA) in a volume of 50 μL of RPMI medium supplemented with 10% of fetal bovine serum (FBS) and 50 μg/mL of gentamycin. In addition, serial dilutions of the mutated IL-15 from 6 μg/mL, were performed in 30 μL of supplemented RPMI medium. Dilutions of IL-15Mut were co-incubated with 20 μL of 300 μg/mL native IL-15. Then, previously washed CTLL-2 cells with RPMI medium were added in amounts of 5×10³ cells/well in 50 μL. The plate was incubated for 72 h at 5% CO₂, 37° C. and 98% relative humidity. Cell viability was determined by MTT staining. IL-15Mut was not biologically active, as it did not induce the proliferation of CTLL-2 cells, unlike recombinant IL-15 (SEQ ID No. 2) and native IL-15, which stimulate the proliferation in a dose-dependent manner (FIG. 2).

### Example 4. Immunization Scheme in Green Monkeys

The animals (green monkeys Chlorocebus sabaeus) were divided into 6 groups of 3 animals each one. A placebo group which received saline phosphate buffer (PBS) solution in aluminum hydroxide adjuvant (Brenntag Biosector, Denmark), a group immunized with 200 μg of the polypeptide identified as SEQ ID No. 2 (IL-15 group), and two other groups, which received 200 μg or 350 μg of the polypeptide identified as SEQ ID No. 1 combined with aluminum hydroxide at a concentration of 1.8 mg/mL. In addition, two other experimental groups immunized with 200 μg of the polypeptide identified as SEQ ID No. 1 or SEQ ID No. 2 in Montanide™ ISA-51 adjuvant (50:50 v/v) were included. Administration of the immunogen was performed subcutaneously at several sites of the interscapular region in a total volume of 0.5 mL. Three immunizations were performed, spaced 15 days between the first and second, and 2 months between the second and third. Blood samples were collected before beginning the scheme (pre-immune serum) and 15 days after the second and third immunization. Complement was inactivated by incubating the sera at 56° C. for 30 min. Vital signs, temperature, heart rate and body weight were registered along the whole scheme.

### Example 5. Determination of Anti-IL-15 Antibodies in Serum from Immunized Animals

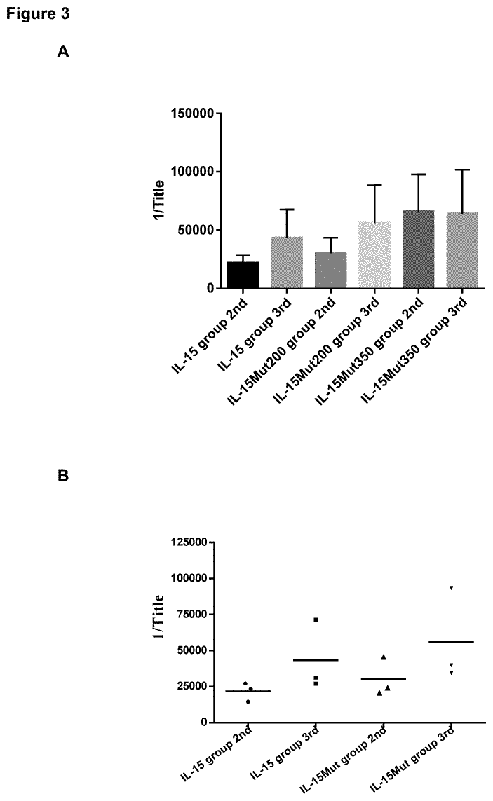

Specific Abs titers against IL-15 were measured through an indirect ELISA, where EIA 96-well plates (Costar, USA) were coated for 16 h at 25° C. with 1 μg/mL of polypeptide identified as SEQ ID No. 2. The plates were blocked with 1% bovine serum albumin (BSA, Sigma-Aldrich, USA) in PBS for 90 min at 37° C. followed by 3 washes with 0.05% Tween 20 in PBS. PBS, 0.05% Tween 20 and 0.01% BSA-diluted sera (starting dilution 1:5000) were added to wells in a volume of 100 μL and incubated for 90 min at 37° C. Wells were then washed 3 times and incubated with anti-human IgG (Fc specific)-peroxidase Ab (Sigma, USA) diluted 1: 10 000 in PBS. After incubating for 1 h at 37° C., the plates were washed 5 times and incubated with 100 μL of substrate solution. The reaction was stopped at 15 min by adding 50 μL of 0.5 M sulphuric acid solution and the absorbance at 450 nm was measured with an ELISA plate reader. Ab titer was considered as the highest serum dilution yielding at least twice the value of the optical density (OD) at 450 nm of the pre-immune serum from each animal. The data were processed using the GraphPad Prism program v6.05 (GraphPad Software, Inc.). The results obtained are shown in FIG. 3; which indicates that active immunization with the mutated polypeptide of IL-15 (corresponding to SEQ ID No. 1) generates an anti-IL-15 antibody response both when using alumina or Montanide™ as adjuvant.

### Example 6. Determination of the Neutralizing Capacity of Sera from Immunized Animals

To evaluate the neutralizing capacity of sera the CTLL-2 cell proliferation assay was performed. This IL-2-dependent cell line also proliferates in presence of IL-15 (maximum proliferation corresponds to cells incubated with 300 pg/mL of native IL-15), whereas in the absence of the cytokine the cells do not proliferate (minimum: cells grown in culture medium without IL-15). In this assay, the molecules that bind to IL-15 and impede the signal translation through its receptor are detected because they inhibit the proliferation of this cell line.

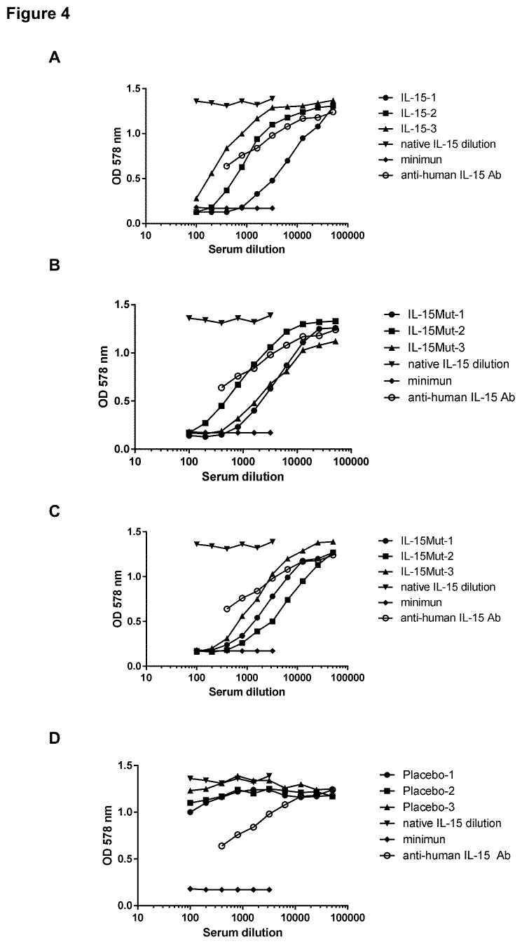

To evaluate the neutralizing capacity of antibodies from immune monkeys, twofold serial dilutions of heat-inactivated sera (starting dilution 1:100) were performed in 96-well plates in a volume of 30 μL of RPMI medium supplemented with 10% FBS and 50 μg/mL of gentamycin. Then, 300 μg/mL of native human IL-15 (R&D, USA) in a volume of 20 μL was added to each well and the plate was incubated at 37° C. for 30 min. Afterwards, previously washed CTLL-2 cells were added in amounts of 5×10³ cells/well in 50 μL and the plate was incubated for 72 h at 5% CO₂, 37° C. and 98% of relative humidity (Rodriguez Y., et al. Biotecnol. Apl. 2014; 31: 291-6). MAB2471(R&D, USA), a neutralizing anti-human IL-15 Ab, was used as a positive control at starting concentration of 1 μg/mL (twofold serial dilutions). Cell viability was determined by MTT staining. The neutralizing titers of sera were expressed as the dilution of sera that is required for inhibiting the proliferation by at least 50% (ID₅₀) and were calculated using the Curve Expert Program version 1.3.8.0. FIG. 4 shows the inhibitory effect of serum on native IL-15-induced proliferation in CTLL-2 cells. These sera were isolated from animals immunized with the polypeptide of SEQ ID No. 1 (IL-15Mut) and SEQ ID No. 2 (recombinant IL-15) using aluminum hydroxide as adjuvant. ID₅₀ values of serum from the individual animals within each experimental group (3 animals per group) are shown in Table 1. When comparing the neutralization titers of the animals receiving the same dose (200 μg) of IL-15Mut (SEQ ID No. 1) and recombinant IL-15 (SEQ ID No. 2), they were found to be higher for the mutated cytokine.

TABLE 1
ID₅₀ values of serum from animals corresponding
to 15 days after the third immunization.

| Immunogen/Dose | Animals | Neutralization Titers (ID₅₀) |
|---|---|---|
| IL-15/200 μg | 1 | 1946 |
| IL-15/200 μg | 2 | 859 |
| IL-15/200 μg | 3 | 280 |
| IL-15Mut/200 μg | 1 | 3547 |
| IL-15Mut/200 μg | 2 | 809 |
| IL-15Mut/200 μg | 3 | 3238 |
| IL-15Mut/350 μg | 1 | 2273 |
| IL-15Mut/350 μg | 2 | 5218 |
| IL-15Mut/350 μg | 3 | 1171 |

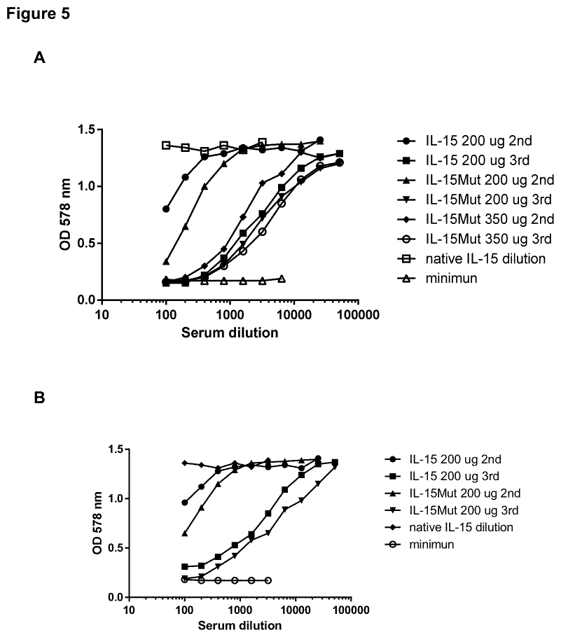

FIG. 5 shows the neutralizing capacity in CTLL-2 cells of pool of sera from animals immunized with IL-15Mut and recombinant IL15. The evaluated sera correspond to 15 days after the second and third immunization, using aluminum hydroxide or Montanide™ as adjuvant. It is observed a neutralizing effect of sera after the third immunization in animals immunized with 200 μg of IL-15Mut and recombinant IL-15. However, the sera corresponding to the dose of 350 μg showed a neutralizing effect at 15 days after the second administration.

Table 2 summarizes the ID₅₀ values of the pool of sera corresponding to 15 days after the second and third immunization, showing the neutralizing titers for the three experimental groups. The immunization with a higher dose (350 μg) of IL-15Mut (SEQ ID No. 1) generates a greater neutralizing response at 15 days after the second immunization, when alumina is used as adjuvant. This is an advantage taking into account that the desired effect could be achieved with fewer administrations. The dose of 200 μg was administrated in combination with Montanide™ as adjuvant. In this case, the ID₅₀ values for IL-15Mut group are also higher than those achieved for recombinant IL-15 group after the second and third inoculations.

TABLE 2
ID₅₀ values of the pool of sera corresponding to
15 days after the second and third immunization.

| | Number of | | Neutralization |
|---|---|---|---|
| | immuniza- | | titers |
| Immunogen/Dose | tions | Adjuvant | (ID₅₀) |
| IL-15/200 μg | 2 | Aluminum hydroxide | 80 |
| IL-15Mut/200 μg | 2 | Aluminum hydroxide | 206 |
| IL-15Mut/350 μg | 2 | Aluminum hydroxide | 1308 |
| IL-15/200 μg | 3 | Aluminum hydroxide | 1256 |
| IL-15Mut/200 μg | 3 | Aluminum hydroxide | 2836 |
| IL-15Mut/350 μg | 3 | Aluminum hydroxide | 3576 |
| IL-15/200 μg | 2 | Montanide ™ ISA-51 | 50 |
| IL-15Mut/200 μg | 2 | Montanide ™ ISA-51 | 175 |
| IL-15/200 μg | 3 | Montanide ™ ISA-51 | 1154 |
| IL-15Mut/200 μg | 3 | Montanide ™ ISA-51 | 1952 |

1) A vaccine composition characterized in that it contains a polypeptide which comprises a mutant of the human Interleukin-15 (IL-15) identified as SEQ ID No. 1, at least one vaccine adjuvant and pharmaceutically acceptable excipients or carriers.

2) A vaccine composition of claim 1 wherein the polypeptide comprising the mutant of IL-15 identified as SEQ ID No. 1 is obtained by recombinant DNA technology.

3) A vaccine composition of claim 1 wherein the polypeptide comprising the mutant of IL-15 identified as SEQ ID No. 1 is a fusion polypeptide.

4) A vaccine composition of claim 3 wherein the fusion polypeptide comprises T epitopes.

5) The use of a polypeptide that it comprises a mutant of the human IL-15 identified as SEQ ID No. 1 to manufacture a medicament for the active immunotherapy of diseases associated with IL-15 overexpression.

6) The use of claim 5 characterized in that the disease associated with IL-15 overexpression is an autoimmune disease selected from the group consisting of rheumatoid arthritis (RA), Crohn's disease, ulcerative colitis and psoriasis.

7) The use of claim 5 characterized in that the disease associated with IL-15 overexpression is a hematologic neoplasia.

8) The use of claim 7 characterized in that the hematologic neoplasia is a leukemia or lymphoma.

9) A method for the treatment of diseases associated with IL-15 overexpression characterized in that it comprises the administration to a subject that needs it of a therapeutically effective amount of a vaccine composition comprising a polypeptide that it comprises a mutant of human IL-15 identified as SEQ ID No. 1.

10) The method of claim 9 characterized in that the disease associated with IL-15 overexpression is an autoimmune disease selected from the group consisting of RA, Crohn's disease, ulcerative colitis and psoriasis.

11) The method of claim 9 characterized in that the disease associated with IL-15 overexpression is a hematologic neoplasia.

12) The method of claim 11 characterized in that the hematologic neoplasia is a leukemia or lymphoma.

13) The method according to claim 10 characterized in that the individual received additionally anti-inflammatory drugs or antagonists of other cytokines.

14) The method according to claim 11 characterized in that the individual received additionally at least one pharmaceutically acceptable cytostatic.


SEQUENCE LISTING (4 sequences)

SEQ ID NO: 1 - protein, 114 aa, Artificial Sequence
  Description of the artificial sequence Mutant of Human Interleukin-15
NWVNVISSLKKIEDLIQSMHIDATLYTESDVHPSCKVTAMKCFLLELQVISLESGDASIHDTVENLIILANNSLSSNGNVTESGCKECEELEEKNIKEFLQSFVHIVSMFINTS

SEQ ID NO: 2 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 3 - DNA, 42 bp, Artificial Sequence
  Description of Artificial Sequence 5′ End of Oligonucleotide of mutant of Interleukin-15
catgccatggcaaactgggtgaatgtaataagttctttgaaa

SEQ ID NO: 4 - DNA, 40 bp, Artificial Sequence
  Description of Artificial Sequence 3′ End of Oligonucleotide of mutant of Interleukin-15
cgggatcccgttaagaagtgttgatgaacatagagacaat